(19) United States
(12) Patent Application Publication
(10) Pub. No.: US 2006/0229657 A1
(43) Pub. Date: Oct. 12, 2006

(54) Single operator exchange embolic protection filter

(76) Inventors: Lawrence D. Wasicek, San Jose, CA (US); Richard J. Renati, Los Gatos, CA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/093,662

(22) Filed: Mar. 30, 2005

(51) Int. Cl. A61M 29/00

(52) U.S. Cl. 606/200

(57) ABSTRACT

Systems and methods for transporting and deploying intravascular devices through a body lumen are disclosed. An intracorporeal device in accordance with an exemplary embodiment of the present invention can include an elongate member with a proximal portion defining first, second and third lumens and a distal portion defining a containment lumen. A filter assembly can be disposed within the containment lumen, with a filter wire attached to the filter assembly and extending through the first lumen. A guidewire can be passed through a distal guidewire lumen that extends through the filter assembly. The guidewire can extend through the distal guidewire lumen and through the second or third lumen. With the filter assembly disposed in the elongate member, the guidewire can then be advanced through a patient's vasculature, the filter delivery system advanced through the vasculature over the guidewire and the filter assembly deployed from the containment lumen.

### FIELD OF THE INVENTION

The present invention relates generally to the field of intracorporeal devices. More specifically, the present invention pertains to systems and methods for transporting and exchanging intravascular devices such as embolic filters within a body lumen.

### BACKGROUND OF THE INVENTION

Guidewires are frequently used to advance intravascular devices to various locations within the body such as an artery or vein. Examples of therapeutic procedures employing such devices include percutaneous transluminal coronary angioplasty (PTCA), percutaneous extraction atherectomy, and stent placement. In a PTCA procedure, for example, a guidewire is percutaneously inserted into a patient's body, and then advanced to a target site where a stenosis or other occlusion is located. Once in place, an angioplasty catheter having an inflatable balloon is advanced along the guidewire and positioned across the site of the stenosis to be dilated. The inflatable balloon is then inflated, causing some embolic material to dislodge from the wall of the vessel and flow downstream.

To prevent the escape of embolic material dislodged during the therapeutic procedure, an embolic protection filter can be advanced to a location distal the target site and deployed to capture emboli present within the blood stream. These devices typically comprise a support structure coupled to a filter mesh or membrane that captures embolic material such as plaque and thrombus, while permitting the perfusion of blood through the vessel. The embolic protection filter may be configured to self-deploy within the vessel when actuated, and may be configured to radially collapse within a catheter or other delivery device to facilitate transport through the body.

During interventional vascular procedures such as angioplasty, atherectomy, thrombectomy and stenting, access to the lesion is often difficult due to the tortuous nature of the vasculature. To access the site of the lesion to be treated, the physician may advance an elongated wire such as a guidewire to a location within the vessel distal the lesion. Such guidewires are typically about 0.014 inches in diameter, and vary in stiffness along their length. Since such guidewires often have a relatively small profile in comparison to other intravascular devices such as angioplasty catheters or stent delivery catheters, the ability to advance an intravascular device across the site of the lesion may be improved by using more conventional guidewires.

### SUMMARY OF THE INVENTION

The present invention relates generally to the field of intracorporeal devices. More specifically, the present invention pertains to systems and methods for transporting and exchanging intravascular devices within a body lumen. One exemplary embodiment of the present invention comprises an elongated member with a proximal portion and a distal portion. The proximal portion has first and second ports and defines first and second lumens. The distal portion has a distal port and defines a containment lumen. The first and second lumens extend from a proximal end of the containment lumen to the first and second ports, respectively. The first port is at or near the proximal end of the elongate member, and the second port is distal of the first port.

Another example embodiment includes a filter delivery system according to the last paragraph with the addition of a third lumen and a third port in the proximal portion of the elongate member. The third lumen extends from the proximal end of the containment lumen to the third port, and the third port is located distal of the first port. The third port can be located at the same position along the elongate member as the second port, and can be located on the opposite side of the elongate member from the second port.

Another exemplary embodiment includes a filter delivery system in conjunction with a filter assembly. Several exemplary filter delivery systems are given in the previous two paragraphs. The filter assembly can comprise a filter, a filter body that defines a distal guidewire lumen, and a filter wire that is connected to the filter body. The filter body also has distal and proximal ports, with the distal guidewire lumen connecting these two ports. The filter wire is sufficiently long to pass through the first lumen and out the first port. The filter assembly is shaped and configured to fit within the containment lumen, and the filter is maintained in a closed position when in the containment lumen. In one embodiment, the filter is predisposed to assume the open position when it is outside the containment lumen.

An exemplary method of the current invention comprises the step of providing a filter delivery system, such as, but not limited to, a filter delivery system in accordance with any of the filter delivery systems described above. The method includes the step of providing a filter assembly, such as, but not limited to, a filter assembly as described above. The filter assembly is placed inside the containment lumen, with the filter wire extending proximally through the first lumen. A guidewire proximal end is fed through the distal guidewire lumen and through the second lumen (or, if there is a third lumen, the guidewire can pass through either the second or third lumens). The guidewire distal end is then fed through a patient's vasculature to a region of interest, and the elongate member along with the filter assembly is fed over the guidewire to the region of interest. The filter assembly can then be deployed from the containment lumen. The deployment can occur by moving the filter wire distally relative to the elongate member.

In another embodiment, the current invention can include a stylet. The stylet has a distal end and a proximal end, and the distal end can be shaped in order to make it easy to grasp. The stylet can be fed through the distal guidewire lumen and can exit the proximal side of the filter body. Thus, as the filter assembly is fed into the containment lumen, the filter wire can be fed into the first lumen and the proximal end of the stylet can be fed into the second or third lumens. The stylet can then be removed and the distal guidewire lumen is aligned with the second or third lumens. The proximal end of the guidewire can then be passed through the distal guidewire lumen and through the second or third lumens.

The above summary of some embodiments is not intended to describe each disclosed embodiment or every implementation of the present invention. The Figures, and Detailed Description which follows, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A is a perspective view of an embodiment of the current invention with an intravascular device contained within a delivery system;

FIG. 1B is a perspective view of another embodiment of the current invention with a filter assembly deployed from a delivery system;

FIG. 2 is a cross-sectional view of a triple-lumen design for a delivery system;

FIG. 2A is a cross-sectional view along line A-A of an embodiment of the triple lumen design for a delivery system;

FIG. 2B is a cross-sectional view along line A-A of an alternate embodiment of the triple lumen design for a delivery system;

FIG. 3 is a cross-sectional view of an alternate embodiment of a delivery system;

FIG. 4 is a cross-sectional view of an embodiment of a delivery system with a filter assembly disposed within the delivery system;

FIG. 5 is a cross-sectional view of a dual lumen delivery system;

FIG. 5A is a cross-sectional view of a dual lumen delivery system with a filter assembly disposed within the delivery system;

FIG. 6A is a cross-sectional view of a delivery system using a stylet;

FIG. 6B is another cross-sectional view of a delivery system using a stylet;

FIG. 6C is another cross-sectional view of a delivery system using a stylet;

FIG. 7 is a cross-sectional view of one embodiment of a filter assembly;

FIG. 8 is a cross-sectional view of an alternate embodiment of a filter assembly;

FIG. 9 is a cross-sectional view of an alternate embodiment of a filter assembly; and

FIG. 10 is a cross-sectional view of an alternate embodiment of a filter assembly.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, materials and manufacturing processes are illustrated for the various elements, those skilled in the art will recognize that many of the examples provided have suitable alternatives that may be utilized.

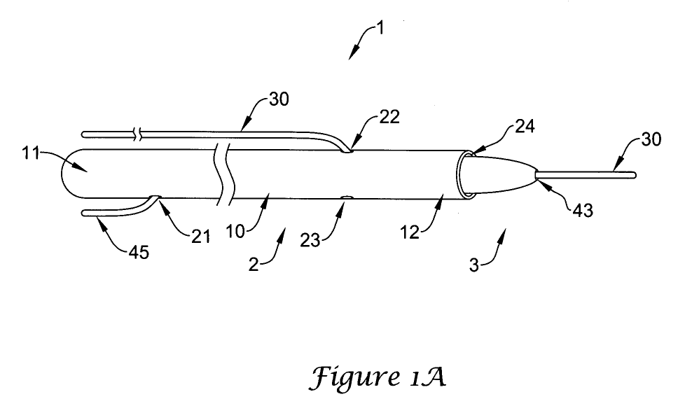

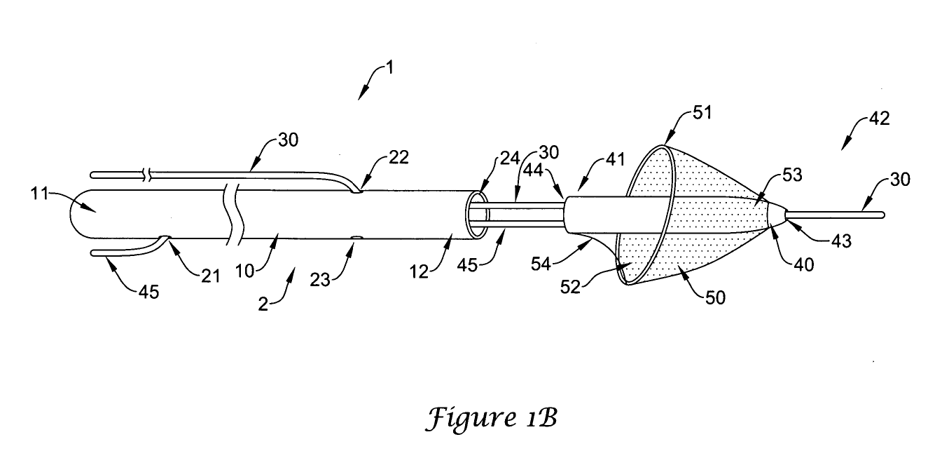

FIG. 1A is a perspective view of a device 1 for deploying intravascular devices, such as embolic filters. The device 1 comprises a delivery system 2 and a filter assembly 3. The delivery system 2 comprises an elongate member 10, which has a distal end 12 and a proximal end 11. The elongate member 10 further comprises a filter wire port 21, a first guidewire port 22 and a second guidewire port 23. The distal end of the elongate member also defines a distal port 24.

In FIG. 1A, the filter assembly 3 is disposed within the delivery system 2, and FIG. 1B shows the filter assembly 3 in the deployed state, outside of the delivery system 2. The filter assembly 3 can be placed within the delivery system through distal port 24. The filter assembly 3 comprises a filter body 40, an embolic protection filter 50, and a filter wire 45. The filter body 40 comprises a distal port 43 and a proximal port 44, and the filter body 40 defines a distal guidewire lumen connecting these two ports (the lumen is shown in subsequent figures).

A guidewire 30 can pass through the distal guidewire lumen, through another lumen defined by the elongate member 10 (again, the lumens of the elongate member are described later), and out the guidewire port 22. In the alternative, the guidewire could pass through an alternate lumen in the elongate member 10 and out guidewire port (22 or 23). The filter wire 45 is attached to the filter body 40, and can extend back through the distal port 24, through a lumen defined by elongate member 10 and out the filter wire port 21.

The embolic protection filter 50 can include a filter mesh or membrane operatively coupled to a support system that comprises a suspension arm 54 and a support hoop 51. The support system may comprise a shape-memory material such as a nickel-titanium alloy, allowing the support hoop 51 to bend and flex while maintaining its original shape. As such, the filter may be predisposed to assume an open, deployed state.

As shown in FIG. 1B, the filter wire 45 can be attached to the filter body proximal end 41. The filter wire 45 can be firmly attached, or the filter body 40 and the filter wire 45 can be rotatable with respect to one another. If the filter body 40 and the filter wire 45 are rotatable with respect to one another, the end of the filter wire 45 can have an enlarged portion at its distal end that fits within, and can rotate within, a cavity of the filter body 40. Alternatively, filter wire 45 can be attached to filter body 40 by means of a shrink-fit, adhesive, soldering, welding, crimping, or other suitable attachment means.

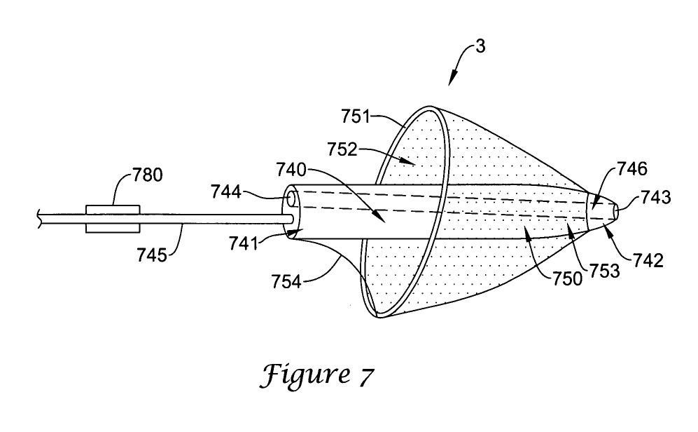

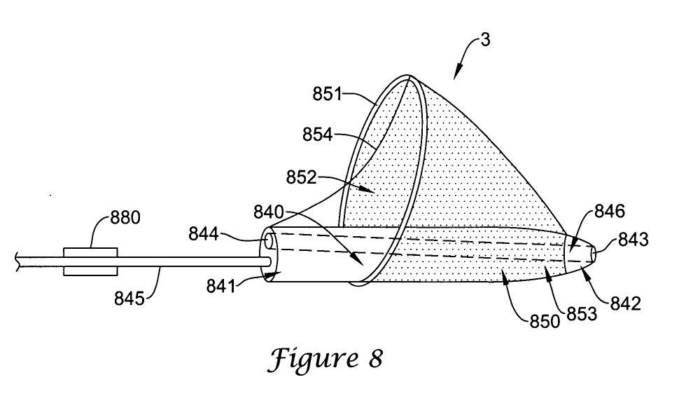

Further elaboration of the filter assembly designs is given later, for example in the description of FIGS. 7-10.

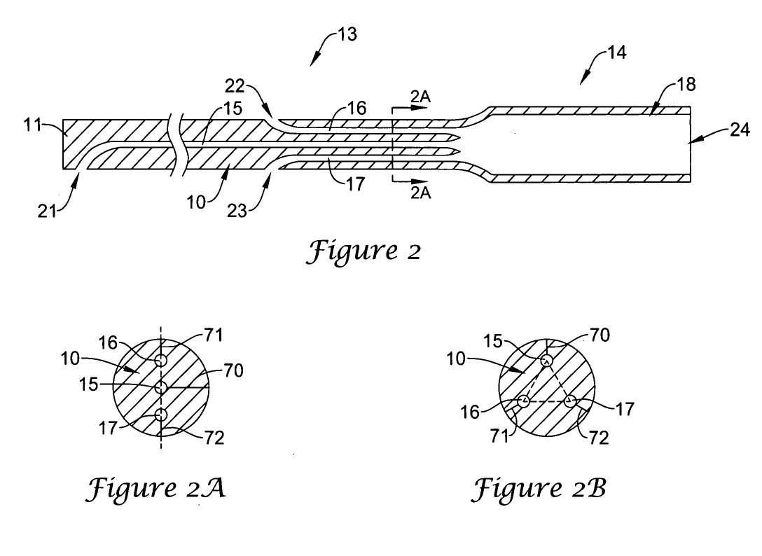

A cross-section of an exemplary embodiment of a delivery system is illustrated in FIG. 2. In this figure, the delivery system comprises an elongate member 10 with a proximal portion 13 and a distal portion 14. The proximal portion 13 defines first, second and third lumens (15, 16, 17) and first, second and third ports (21, 22, 23). The distal portion 14 defines a containment lumen 18 and a distal port 24. The distal and proximal portions (13, 14) can be either separate structures that are attached or they can be integrally part of the same structure. The two portions can be made of the same material, or from different materials. The containment lumen 18 can extend from the junction between the distal and proximal portions (13, 14) to the distal port 24. In this embodiment, a filter wire lumen 15 extends from the proximal end of the containment lumen 18 to the first port 21. The first port 21 is located near the elongate member proximal end 11. In the alternative, the first port 21 can comprise an opening through the elongate member proximal end 11. The second and third lumens (16, 17) extend from the containment lumen proximal end to the first and second ports (22, 23), respectively. In this embodiment, the second and third ports are located distally of the first port, and are located on opposite sides at approximately the same position along the elongate member.

In FIG. 2A, a cross-section along the line A-A of the elongate member is shown. In this example, the three lumens (15, 16, 17) are aligned. That is, the centers of the cross-sections of the lumens are substantially in the same plane. In the alternative, other configurations of the lumens are contemplated, such as the triangle shape shown in FIG. 2B. In FIG. 2B, connecting the center of the cross-sections of each lumen (15, 16, 17) substantially forms a triangle. This triangle is shown as an equilateral triangle, but the formation of non-equilateral triangles is also contemplated.

The lumens (15, 16, 17) are shown in FIGS. 2A and 2B having a round cross-section. However, the cross-section of the lumens could have other cross-sections, such as oval, rectangle, square, triangle, polygonal, or the like, or combinations thereof. The shape of the lumens (15, 16, 17) can be chosen to match the shape of a particular filter wire or guidewire that may be used. The shape and size of the lumens (15, 16, 17) could be chosen to allow for rotation of the filter wire or guidewire within the lumen, or rotation could be prevented by the interaction between the shape of the lumen and the wire. For example, a triangular shaped guidewire fitting tightly within a triangular shaped lumen may not be able to readily rotate. Also, coatings could be used on the inside of the lumens (15, 16, 17). For example, a lubricious coating could be used in order to lower friction and facilitate movement of wires within the lumens, or a pliable coating could be used to provide a seal between the wire and the wall of the lumen.

Also shown in FIGS. 2A and 2B are slits (70, 71, 72). The slits (70, 71, 72) can facilitate the removal of a filter wire or guidewire from the device. For example, a slit or scoring 70 can be made in the wall of the elongate member along all or a portion of the length of the elongate member from the filter wire port to the distal end of the elongate member. This can facilitate the elongate member 10 being peeled away from the filter wire 45 during a procedure without having to run the elongate member 10 all the way off of the end of the filter wire 45. Similarly, a slit or score (71, 72) can be made in the wall of the elongate member along all or a portion of the length of the elongate member from the respective guidewire port to the distal end of the elongate member. These slits can facilitate removal of a guidewire that might be disposed in the second or third lumens (16, 17).

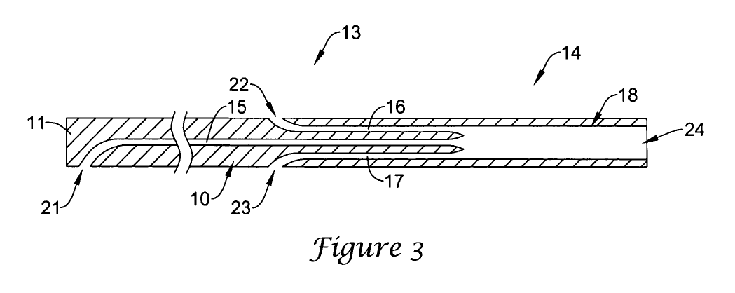

In FIG. 2, the distal portion 14 is shown having a larger outer diameter than the proximal portion 13. Such an arrangement can allow for a larger containment lumen 18 in which to fit large sized intravascular devices. In FIG. 3, the distal portion 14 is shown as having a similar outer diameter compared to the proximal portion 13. As an alternative, the distal portion 14 may have a smaller outer diameter compared to the proximal portion 13.

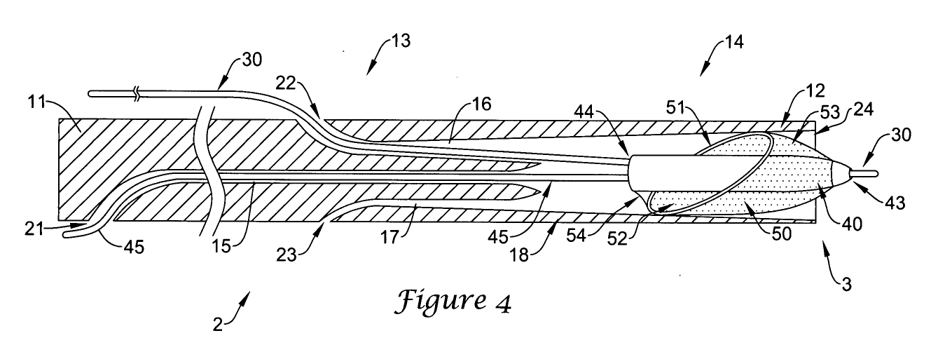

Refer now to FIG. 4, which is a cross-section of a device 1 for deploying intravascular devices. The device comprises a delivery system 2 and a filter assembly 3. The delivery system 2 is similar to the delivery system described in FIG. 3, although other triple lumen delivery systems could also be used in this example. Also, the filter assembly 3 is similar to the filter assembly described in FIG. 1B, although other filter assemblies can be used in this example.

As shown in FIG. 4, the filter assembly 3 is disposed within the delivery system 2. The filter assembly 3 can fit inside of the containment lumen 18 of the delivery system 2. The filter wire 45 that is attached to the filter body 40 extends proximally through the filter wire lumen 15 and out of the filter wire port 21. In addition, the filter body 40 defines a distal guidewire lumen (for example, as shown in later Figures), and the distal guidewire lumen connects the filter body proximal 44 and distal 43 ports. As shown, a guidewire 30 can pass through a distal port 43, the distal guidewire lumen, and the proximal port 44 and through the first proximal guidewire lumen 16 and out the first guidewire port 22. In this way, the device 1 can be a single-operator exchange device.

In the alternative, rather than passing through the first proximal guidewire lumen 16 and the first guidewire port 22, the guidewire 30 could pass through the second proximal guidewire lumen 17 and the second guidewire port 23. The fact that the guidewire 30 could pass through either the first or second proximal guidewire lumens (16, 17) allows the guidewire to more freely pass through the filter assembly 3 and the delivery system 2. With more than one possible path to travel, the guidewire 30 may be less likely to get entangled with other elements of the device 1, such as the filter wire 45.

As an alternate embodiment, the triangular positioning of the three lumens as mentioned with respect to FIG. 2B may also allow for easy passage of the guidewire 30. In a similar manner, the two proximal guidewire lumens (16, 17) in a triangular design can allow for convenient and multiple passageways through which the guidewire can pass. As mentioned above, this can prevent the guidewire 30 from getting entangled with the other elements of the device 1.

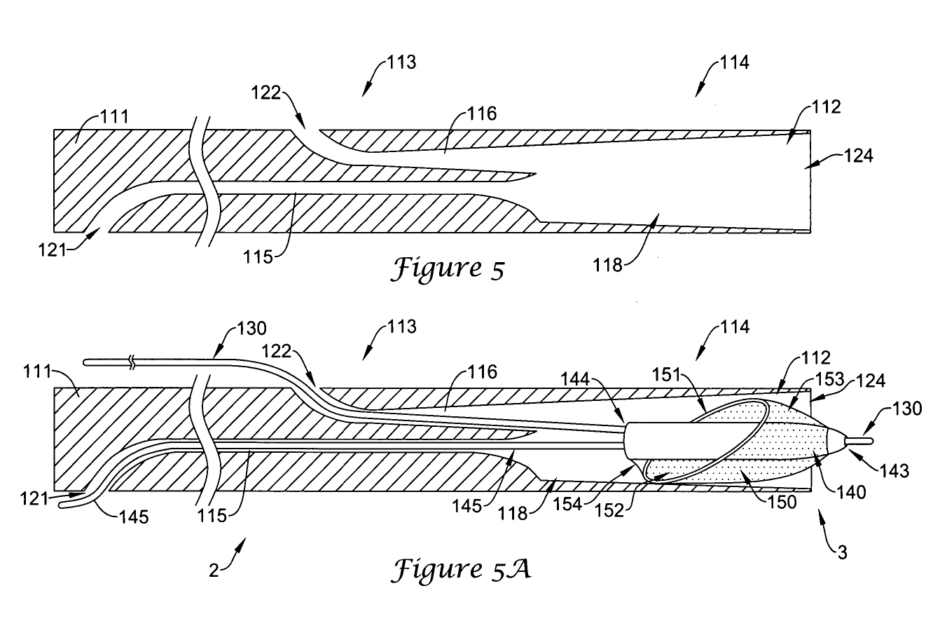

FIG. 5 shows an alternate embodiment of the current invention. This figure shows a delivery system 2 comprising a proximal portion 113 and a distal portion 114. The proximal portion 113 defines first and second lumens (115, 116) and first and second ports (121, 122). The distal portion 114 defines a containment lumen 118 and a distal port 124.

FIG. 5A shows a dual lumen delivery system 2 (for example, a dual lumen delivery system like the one from FIG. 5), with a filter assembly 3 disposed in the containment lumen 118. In this example, similar reference numbers indicate similar structure. Like the triple lumen delivery systems described above, this example embodiment can facilitate the use of a variety of guidewires. As shown in FIG. 5A, the filter assembly 3 can be placed in the containment lumen 118, and a guidewire 130 can then be fed through the distal port 143, the distal guidewire lumen (for example, as shown in later figures) and the proximal port 144 and through the proximal guidewire lumen 116 and the guidewire port 122. This design can allow any guidewire to be used in conjunction with the filter assembly 3 and the delivery system 2 as long as it is sized and shaped to fit through the guidewire lumens.

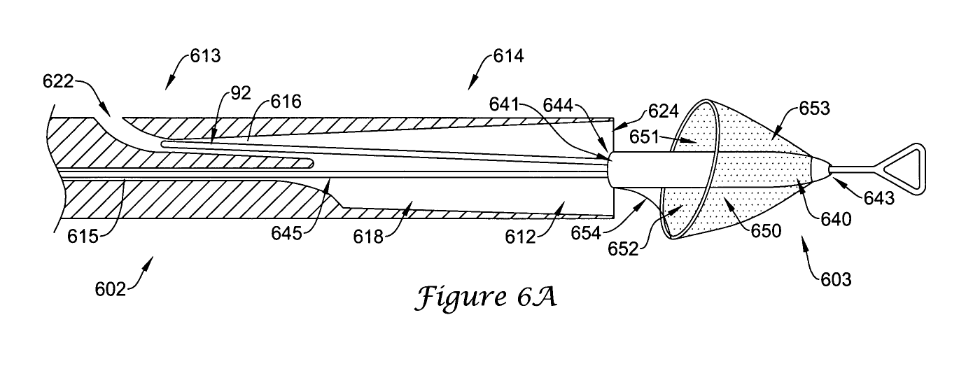

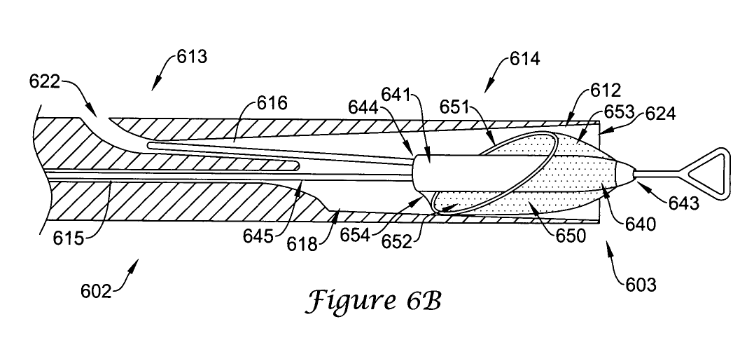

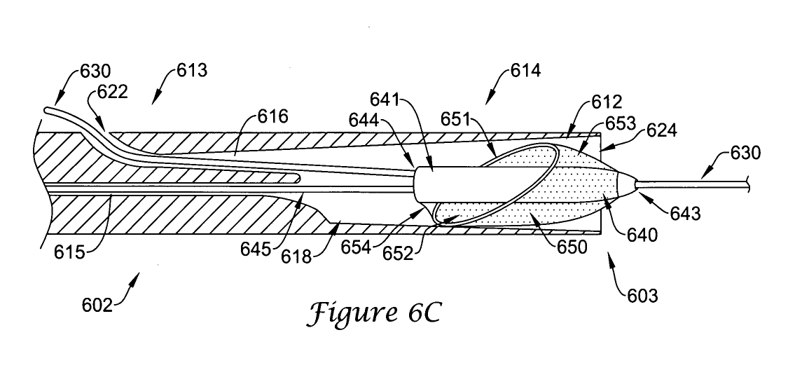

FIGS. 6A-6C show an alternate embodiment of the current invention. This embodiment shows a dual lumen delivery system 602 in conjunction with a filter assembly 603 and a stylet 90. It is contemplated that the filter assembly design that is shown can be used. However, any other suitable filter assembly design that is described in this application may also be used.

The stylet 90 has a distal end 91 and a proximal end 92, and is sized to fit through the distal guidewire lumen (for example, as shown in later figures) of the filter body 640 and through the proximal guidewire lumen 616. As shown in FIG. 6A, the stylet 90 can be inserted into the distal guidewire lumen so that the stylet proximal end 92 extends from the filter body proximal end 641. The filter assembly 603 can then be inserted into the containment lumen 618. The stylet distal end 91 can be shaped or bent in order to provide for a shape that is easily grasped, such as that shown in FIGS. 6A-6C. When the filter assembly 603 is being inserted into the containment lumen 618, the filter wire 645 can be fed into the filter wire lumen 615 and the stylet proximal end 92 can be fed into the proximal guidewire lumen 616. The stylet could also be further fed through port 622. The stylet 90 can then be removed.

In this example, the filter body 640 will be aligned such that a guidewire 630 that is inserted into the distal port 643 and through the distal guidewire lumen 646 can exit the proximal port 644 and enter the proximal guidewire lumen 616. FIGS. 6A-6C show the use of a stylet 90. FIG. 6A shows the filter wire 645 and the stylet proximal end 92 entering the filter wire lumen 615 and the proximal guidewire lumen 616, respectively. FIG. 6B shows the filter assembly 603 disposed within the containment lumen 618, with the stylet 90 still in place. The stylet 90 can then be removed, and a guidewire 630 inserted into the distal port 643, through the distal guidewire lumen, into the proximal guidewire lumen 616 and out the guidewire port 622, as shown in FIG. 6C. Thus, the stylet 90 can be used to facilitate the use of a guidewire 630.

Similar to the above description of the use of a stylet with a dual lumen delivery system, a stylet could also be used with a triple lumen delivery system. In such use, when the filter assembly is inserted into the containment lumen, the filter wire can be fed into the filter wire lumen and the proximal end of the stylet can be fed into either of the guidewire lumens, often whichever guidewire lumen most easily lines up with the proximal end of the stylet. Similarly, the stylet could then be removed, and a guidewire inserted through the filter body and through one of the proximal guidewire lumens and out a guidewire port.

The stylet that is used can be made of any suitable material, including metals, metal alloys, polymers, and the like. The cross-sectional shape of the stylet can be round in shape, or can be any other suitable shape such as oval, rectangle, square, triangle, polygonal, or the like. The stylet can be of solid cross-section, hollow, or it can be made of multiple elements, such as a braided construction.

FIGS. 7-9 are detailed drawings of possible embodiments of the filter assembly. In FIG. 7, a detailed drawing of a filter assembly 3 is shown. The filter assembly 3 comprises a filter wire 745, a filter body 740, and a filter 750. The filter wire 745 can consist of any suitable material, including metals, metal alloys, polymers, and the like. One embodiment of the filter wire 745 is made of stainless steel. The cross-sectional shape of the filter wire 745 can be round in shape, or can be any other suitable shape such as oval, rectangle, square, triangle, polygonal, or the like. The filter wire 745 can be of solid cross-section, hollow, or it can be made of multiple elements, such as a braided construction. The filter wire 745 can be of sufficient length to allow the filter wire 745 to pass through the entire length of the filter wire lumen.

The filter wire 745 can be connected to the filter body 740 at or near the filter body proximal end 741. The filter wire 745 can be firmly attached, or the filter body 740 and the filter wire 745 can be rotated with respect to one another. The filter wire 745 can be attached to the center of the filter body 740, or can be offset to one side of the filter body 740, as shown in FIG. 7. If the filter body 740 and the filter wire 745 are rotatable with respect to one another, the end of the filter wire 745 can have an enlarged portion at its distal end that fits within, and can rotate within, a cavity of the filter body 740. Alternatively, filter wire 745 can be attached to filter body 740 by means of a shrink-fit, adhesive, soldering, welding, crimping, or other suitable attachment means.

The devices described in this application may also comprise stoppers on the filter wire, the guidewire, or the stylet, or any combination of the three, in order to control the positioning of the filter wire, the guidewire or the stylet. For example, the filter wire can have an enlargement proximal the filter body. FIG. 7 shows an enlargement as a coil 780 on the filter wire 745. The enlargement 780 can also be a sleeve or a crimp in a wire. This enlargement 780 can prevent the filter assembly from moving too far proximally when loading the filter assembly 3 into the delivery system 2. Similarly, the guidewire can also have an enlargement, for example an enlargement near its distal end. In some applications, this can prevent the guidewire from being pulled proximally out of the filter body. Also, if the filter body were to be retrieved, the guidewire may be able to assist in pulling the filter into a retrieval sheath.

The filter wire and the guidewire can also be shaped and sized to fit within the respective filter wire and guidewire lumens. The filter wire and guidewire can have a round cross-section, or they can have a cross-section that is an oval, rectangle, square, triangle, polygonal, or the like, or any other suitable shape, or a combination thereof. The size and shape of the wire can be chosen to allow the wire to rotate with respect to the elongate member when the wire is disposed in a lumen. As an alternative, the shape and size can be chosen to prevent such rotation. For example, if the wire and the lumen in which it is placed were triangular and the wire was sized to snugly fit within the lumen, the wire may not be able to rotate within the lumen. Also, the size and shape can be chosen to provide a friction fit between the outer surface of the wire and the inner surface of the lumen, or the size and shape could allow for space between these two surfaces.

The filter in FIG. 7 comprises an open end 752, a closed end 753, a mesh or membrane between the open and closed ends, and a filter support structure. The filter open end 752 points in the proximal direction, although it is contemplated that the open end 752 could also point in the distal direction, depending on the desired use.

The mesh or membrane can be operatively coupled to a support system that comprises a support hoop 751. Alternatively, the support system can comprise a suspension arm 754 and a support hoop 751. The support system may comprise a shape-memory material such as a nickel-titanium alloy, allowing the support hoop 751 to bend and flex while maintaining its original shape. As such, the filter may be predisposed to assume an open, deployed state. While the filter open end 752 can be attached to a support structure, the filter closed end 753 can be attached directly to the filter body 740. The suspension arm 754 can be attached to the filter body 740 on one end and can be attached to the support hoop 751 on the other end. Attachment of the suspension arm to the filter body or a support hoop directly to the filter body can be accomplished by any suitable attachment means such as adhesive, brazing, soldering, welding, crimping or any combination(s) thereof.

As shown in FIG. 8, the filter support system can also comprise a support hoop 851, with the support hoop 851 directly attached to the filter body 840, thus disposing the filter 850 concentrically to one side of the filter body 840. In the embodiment of FIG. 8, the support structure can also include the use of a suspension arm 854.

As another alternative, the filter assembly may comprise an inflatable cuff (for example, in place of the support hoop 51) and a lumen extending down the filter wire and in communication with the inflatable cuff. Inflating the inflatable cuff could then deploy the filter 50. In addition, it is contemplated that alternate means of mechanical actuation could be used for deploying the filter.

Referring back to FIG. 7, the filter body 40 defines a distal guidewire lumen 46 that extends from a distal port 43 to a proximal port 44. This distal guidewire lumen 46 is shown as being straight and having a round cross-section, and offset to one side of the filter body proximal end 41. It is also contemplated that the lumen can be curved and that the lumen could have a cross-sectional shape such as oval, rectangle, square, triangle, polygonal, or the like, or any other suitable shape. The distal guidewire lumen may also extend down the center of the filter body. The distal guidewire lumen may include a polymeric liner such as polytetrafluoroethylene (PTFE) to provide a smooth, lubricious interior surface for a guidewire. In addition, the distal guidewire lumen can be lined with a pliable material that will conform around a variety of sizes of guidewires. As such, when the filter assembly is deployed, blood or other fluids cannot readily travel through the distal guidewire lumen, and thus the fluids will travel through the filter and emboli will be captured in the filter. It is also contemplated that the distal guidewire lumen can be slightly larger in diameter near the distal port or near the proximal port or both. This slight enlargement can facilitate the entry of a wire into the lumen.

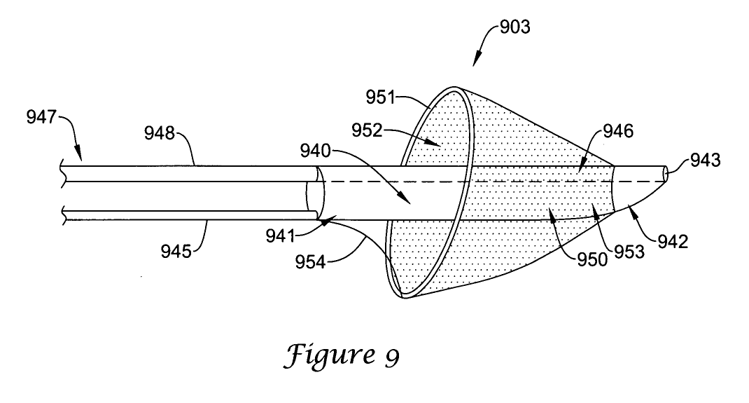

In addition, and as shown in FIG. 9, a distal guidewire lumen extension 948 could extend proximally from the filter body 940. Thus, when the filter assembly 903 is being fed into the containment lumen as mentioned in this application, the distal guidewire lumen extension 948 may be able to line up with a proximal guidewire lumen and facilitate the efficient introduction of a guidewire without the use of a stylet.

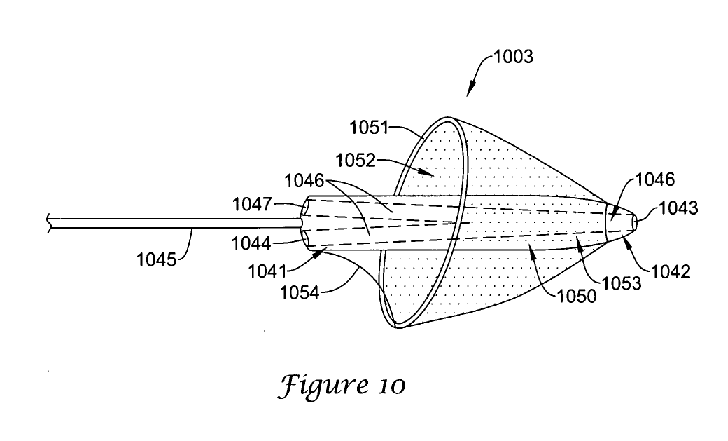

Referring to FIG. 10, it is contemplated that the proximal end of the filter body can have two proximal ports (1044, 1047). In such a case, the distal guidewire lumen 1046 can be bifurcated such that a guidewire being introduced to the distal guidewire lumen 1046 through the distal port 1043 can travel through either proximal port (1047, 1044). Such a bifurcated lumen design in conjunction with a triple lumen delivery system can allow the guidewire to travel through either proximal port (1044, 1047) and into either proximal guidewire lumen.

In general, the filter body can be constructed of any material, such as metals, metal alloys, polymers, and the like, or other suitable materials. For example, the materials of construction for the filter body can allow for a high degree of flexibility for the filter body due to the fact that it can be located on the distal end of the device where flexibility is often desired in order to navigate tortuous vasculature. The filter body could comprise one material, or could be made of several materials, for example several layers of material. The filter body could comprise a tubular structure, a coil, or any other suitable structure. The distal end of the filter body can be generally tapered to allow for efficient navigation through a patient's vasculature. The distal end of the filter body can also be tapered and fit tightly about the guidewire. This tight fit around the guidewire could assist in making the end of the filter body stiffer in order to cross lesions in a patient's vessel. This could facilitate movement through vasculature and prevent material deposits between the guidewire and the filter body.

In addition, the distal end can comprise a soft, atraumatic tip design to prevent damage or perforation of a vessel wall. As such, the distal tip, or the entire filter body, could comprise a pliable material such as a soft plastic. The proximal end of the filter body can also have a tapered shape, which can facilitate the entry of the filter body into the containment lumen. In addition, the filter body is shown having a round cross-section in all of the figures, but can also have other shapes such as oval, rectangle, square, triangle, polygonal, or the like, or any other suitable shape.

The filter body could also be tapered at the proximal end. This tapered profile could facilitate entry of the filter body into the sheath and could also ensure that the proximal end of the filter body does not catch on or damage a patient's vasculature.

The filter body can be formed from an injection mold process utilizing a suitable polymeric material such as polypropylene (PP) or polyvinylchloride (PVC). In other embodiments, the filter body may be formed from different members and/or materials that are coupled together. For example, proximal and distal sections of filter body may be formed of a polymeric member, whereas a middle section of the filter body may comprise a coil or slotted hypotube. The various sections of the filter body can be bonded together by adhesive, welding, crimping, soldering, insert molding, or other suitable bonding technique.

Referring back to FIG. 1A, the elongate member 10 can have a variable flexibility from the distal end 11 to the proximal end 12. The elongate member can be more flexible at the distal end or the proximal end, depending on the desired use. A distal region could be more flexible than a proximal region in order to facilitate navigation through a tortuous path in a patient's vasculature. For example, the cross-section of the elongate member can be smaller on the distal end 12 relative to the proximal end 11. The elongate member can be linearly tapered, tapered in a curvilinear fashion, uniformly tapered, non-uniformly tapered, or tapered in a step-wise fashion. The flexibility can also be varied along the elongate member 10 by adding reinforcement members in order to make portions of the elongate member 10 less flexible or by removing material from portions of the elongate member 10, or both. For example, a coil can be placed at the distal end of the elongate member in order to make the distal tip slightly more durable and facilitate easy crossing of stenosis and easy loading of the filter assembly into the containment lumen.

The elongate member can have a round cross-sectional shape, or it could have a cross-section that is oval, rectangle, square, triangle, polygonal, or the like, or any other suitable shape, or combinations hereof. The elongate member can comprise materials such as metals, metal alloys, polymers, and the like, or other suitable materials, or combinations thereof. The materials can be chosen to impart the desired flexibility characteristics or other characteristics. For example, the elongate member could be made entirely or partially of stainless steel or the nickel-titanium alloy nitinol. It is also contemplated that the elongate member can be constructed of multiple structures, such as one structure for the distal portion and one structure for the proximal portion. As such, the distal portion can be of a different shape or made of different materials in order to impart more or less flexibility on the distal tip. For example, the proximal structure can comprise stainless steel for a relatively stiff proximal portion of the device. The distal end of the device can comprise a more flexible material or a shape memory material, such as a suitably flexible plastic or Nitinol.

Generally, all or a portion of the elongate member can comprise a coil comprising nitinol, stainless steel, or other metal, fibrous material or a polymer, or other suitable material. A polymer can then be disposed about the coil, forming a composite of the coil and the polymer. The polymer can be heat shrunk into the coils in order to form this composite. The polymer that is disposed about the coil can be a thermoplastic polymer, for example Pebax, PET, urethane or other suitable polymers.

The elongate member could also contain a shaping ribbon. The shaping ribbon could be disposed along the length of the elongate member and could also be substantially disposed along the same path as a coil. The shaping ribbon could be used to shape the distal end of the elongate member. For example, the elongate member could originally be straight, and the elongate member could be bent into an alternate shape, with the shaping ribbon allowing the distal end of the device to hold the alternate shape.

In addition, in order to control the flexibility of the device 1, the flexibility of portions of the filter assembly 3 can also be controlled. As mentioned above, the filter body can be made of materials that maintain the flexibility of the distal end of the device 1. The filter body could also be made of relatively stiffer materials in order to facilitate procedures such as crossing of a stenosis.

To further control flexibility along the length of the device 1, the flexibility of the filter wire or the guidewire or both can be varied. The filter wire or the guidewire or both could be more flexible on the distal end relative to the proximal end, or vice versa. The filter wire or the guidewire or both can have a smaller cross-sectional area in the distal region relative to the proximal region. The wires can be linearly tapered, tapered in a curvilinear fashion, uniformly tapered, non-uniformly tapered, or tapered in a step-wise fashion.

The wires can be constructed of any suitable material(s) biocompatible with the body. Examples of such materials include 304 or 316 grade stainless steel, platinum, or nickel-titanium alloy (Nitinol). Nickel-titanium alloy exhibits super-elastic capabilities at body temperature (approximately 37° C.), which permits substantial bending or flexing with a relatively small amount of residual strain. It is contemplated, however, that other materials can be used. For example, in some embodiments, the filter wire and the guidewire may comprise a stainless steel core wire surrounded by a polymeric coating to facilitate smooth transport of other intravascular devices thereon.

The device 1 may have radiopaque elements placed at a position or positions along its length in order to assist in advancement and placement of the device and the filter assembly. For example, a radiopaque coil can be disposed (for example, helically disposed) about the support hoop of the filter support structure and can be used to fluoroscopically judge the placement and deployment status of the embolic filter within the patient. Coils or marker bands can also be placed at other locations along the device. For example, a radiopaque band could be placed near the distal end of the elongate member, or near the distal end of the filter. The marker may be formed of a relatively high radiopaque material such as gold, platinum or tantalum, which can be utilized in conjunction with a fluoroscopic monitor to determine an accurate measure of the location of the embolic filter within the vasculature. Other radiopaque markers could also be placed at intervals along the device in order to view progress of the device through the patient's vasculature.

Additionally, all or portions of the device 1 can include materials or structure to impart a degree of MRI compatibility. For example, all or portions of the device may be made of a material that does not substantially distort the image and create substantial artifacts (artifacts are gaps in the image). Certain ferromagnetic materials, for example, may not be suitable because they may create artifacts in an MRI image. All, or portions of, the device can also be made from a material that the MRI machine can image. Some materials that exhibit these characteristics include, for example, tungsten, Elgiloy, MP35N, nitinol, and the like, and others, or combinations or alloys thereof.

Another embodiment of the current invention can be found in a method for using the devices described herein. The method can include the step of providing a delivery system and a filter assembly. The delivery system can be, but is not limited to, any of the delivery systems described in this application. Likewise, the filter assembly can, but is not limited to, any of the filter assemblies described in this application. The filter assembly can be placed within a containment structure of the delivery system. Further, a guidewire can be provided and fed through a distal guidewire lumen in the filter body of the filter assembly and through a proximal guidewire lumen in the delivery system and out a port. The method can further comprise the step of introducing the guidewire distal end into a patient and advancing the distal end to a position of interest. The combination delivery system and filter assembly can then be advanced over the guidewire to the position of interest. When at the position of interest, the filter assembly can be deployed from the delivery system. The filter wire can be moved distally with respect to the delivery system, thus pushing the filter assembly out of the delivery system.

Once the filter assembly is deployed, the delivery system can be kept just proximal of the filter assembly in order to act as a retrieval sheath after the procedure is complete. In the alternative, the delivery system can be removed during a part of the procedure and reintroduced in order to retrieve the filter assembly, or a separate retrieval device could be used to retrieve the filter assembly.

Further, another method can also include providing a filter assembly with a stylet placed in a distal guidewire lumen. The filter assembly can be placed in a delivery system, using the stylet to align the distal guidewire lumen of the filter assembly with the proximal guidewire lumen of the delivery system. The stylet can then be removed from the distal guidewire lumen, and a guidewire can be introduced to the distal guidewire lumen.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. Changes may be made in details, particular in matters of size, shape, and arrangement of parts without exceeding the scope of the invention. It will be understood that this disclosure is, in many respects, only illustrative.

What is claimed is:

1. An intracorporeal device comprising a filter assembly and a filter delivery member;
the filter delivery member comprising:
a proximal region defining first, second and third lumens and having first, second and third ports; and
a distal region defining a containment lumen and a fourth port;
wherein the first, second and third lumens extend from a proximal end of the containment lumen to the first, second and third ports, respectively;
the filter assembly comprising:
a filter body defining a distal guidewire lumen extending therethrough;
a filter attached to the filter body; and
a filter wire attached to the filter body;
wherein the filter assembly is shaped and configured to fit within the containment lumen.

2. The intracorporeal device of claim 1, wherein the filter wire has a length sufficient to extend through the containment lumen and the longest of the first, second and third lumens.

3. The intracorporeal device of claim 1, wherein the first port is disposed at or near the proximal end of the filter delivery member.

4. The intracorporeal device of claim 1, wherein the second and third ports are disposed distal of the first port.

5. The intracorporeal device of claim 1, wherein the second and third ports are located on opposite sides of the filter delivery member.

6. The intracorporeal device of claim 1, wherein the second and third ports are at the same longitudinal position along the filter delivery member.

7. The intracorporeal device of claim 1, wherein the first, second and third lumens have first, second and third axes, respectively, and the axes are substantially in the same plane.

8. The intracorporeal device of claim 7, wherein the first lumen is between the second and third lumens and the first lumen is substantially coaxial with the filter delivery member.

9. The intracorporeal device of claim 1, wherein a cross-section of the filter delivery member has the first, second and third lumens distributed in a triangle pattern.

10. The intracorporeal device of claim 1, wherein the containment lumen extends distally from the junction between the proximal and distal regions to the fourth port.

11. The intracorporeal device of claim 1, wherein the distal and proximal regions comprise different materials.

12. The intracorporeal device of claim 1, wherein the filter is predisposed to assume an open position when not disposed in the containment lumen.

13. The intracorporeal device of claim 1, wherein the filter comprises a filter membrane operatively coupled to a support hoop, the support hoop forming a mouth for filtering embolic debris within a vessel.

14. The intracorporeal device of claim 13, further comprising a suspension arm with two ends, wherein one end of the suspension arm is attached to the support hoop and the other end is attached to the filter body.

15. The intracorporeal device of claim 1, wherein the filter comprises a filter membrane, wherein the membrane forms a conical shape with an open end and a closed end, wherein the open end is attached to a support hoop and wherein the support hoop and the narrow end of the conical filter membrane are connected directly to the filter body.

16. The intracorporeal device of claim 1, wherein the filter body has a distal port and a proximal port, and the distal guidewire lumen extends therebetween.

17. The intracorporeal device of claim 16, wherein the distal guidewire lumen is substantially straight.

18. The intracorporeal device of claim 1, wherein the filter body has a distal port and two proximal ports, wherein the distal guidewire lumen is bifurcated and the distal guidewire lumen extends from the distal port to both proximal ports such that a guidewire passing through the distal guidewire lumen could pass from the distal port and through either proximal port.

19. The intracorporeal device of claim 1, wherein the filter wire is rotatably attached to the filter body.

20. The intracorporeal device of claim 1, wherein the filter wire is attached to the proximal end of the filter body.

21. The intracorporeal device of claim 1, further comprising a guidewire tube defining an extension lumen, with a distal end of the extension lumen attached to a proximal port of the filter body, wherein the guidewire tube forms a proximal extension of the distal guidewire lumen.

22. The intracorporeal device of claim 1, further comprising a stylet with distal and proximal ends and a handle disposed on the distal end, wherein the stylet proximal end is sized to fit through the distal guidewire lumen and either the first, second or third lumen.

23. The intracorporeal device of claim 1, further comprising a scoring or a cut extending along at least a portion of the filter delivery member between the first port and the fourth port.

24. The intracorporeal device of claim 1, further comprising a scoring or a cut extending along at least a portion of the filter delivery member between the second port and the fourth port.

25. The intracorporeal device of claim 1, further comprising a scoring or a cut extending along at least a portion of the filter delivery member between the third port and the fourth port.

26. The intracorporeal device of claim 1, wherein the proximal region has a first outside diameter and the distal region has a second outside diameter and the second outside diameter is larger than the first.
